(19) United States
(12) Patent Application Publication
(10) Pub. No.: US 2025/0299330 A1
(43) Pub. Date: Sep. 25, 2025

(54) AI-ASSISTED DETECTION OF VASCULAR ANOMALIES IN MEDICAL IMAGES

(71) Applicant: Siemens Healthineers AG, Forchheim (DE)

(72) Inventors: Long XIE, Chesterbrook, PA (US); Bogdan GEORGESCU, Princeton, NJ (US); Eli GIBSON, Lawrenceville, NJ (US); Jing LU, Shanghai (CN); Zhong Yi YAO, Shanghai (CN)

(73) Assignee: Siemens Healthineers AG, Forchheim (DE)

(21) Appl. No.: 19/085,679

(22) Filed: Mar. 20, 2025

(30) Foreign Application Priority Data
Mar. 21, 2024 (DE) 10 2024 202 699.9

(51) Int. Cl. G06T 7/00; G06T 7/11; G06T 7/60; G06T 11/20; G06V 10/25; G06V 10/26; G06V 10/774; G06V 10/82; G06V 20/70; G16H 30/20

(52) U.S. Cl. CPC G06T 7/0012; G06T 7/11; G06T 7/60; G06T 11/203; G06V 10/25; G06V 10/26; G06V 10/774; G06V 10/82; G06V 20/70; G16H 30/20; G06T 2207/20081; G06T 2207/20084; G06T 2207/30104

(57) ABSTRACT

A computer-implemented training data preparation method comprises: receiving an input medical image of vessels of a patient; determining a vessel segmentation from the input medical image; identifying and annotating anatomical landmarks in the vessel segmentation to produce an annotated vessel segmentation; and storing the annotated vessel segmentation as training data. A training method for training neural networks based on the training data and a medical diagnostic method applying trained AI models are also provided.

### CROSS-REFERENCE TO RELATED APPLICATION(S)

The present application claims priority under 35 U.S.C. § 119 to German Patent Application No. 10 2024 202 699.9, filed Mar. 21, 2024, the entire contents of which is incorporated herein by reference.

### FIELD

One or more embodiments of the present invention relate to methods and systems for AI (artificial intelligence) assisted vascular abnormality detection in medical imaging, in particular methods for training an AI model as well as applying the AI model to medical imaging.

### BACKGROUND

Neurovascular abnormalities, including but not limited to occlusions, stenosis, and aneurysms, present significant health risks, necessitating their timely and accurate detection for effective therapeutic intervention and improved patient outcomes. Advanced medical imaging techniques such as Computed Tomography Angiography (CTA) and Magnetic Resonance Imaging such as Magnetic Resonance Angiography (MRA), Time-of-Flight Magnetic Resonance Angiography (MRTOF) and post contrast T1-weighted MRI, have greatly enhanced our ability to visualize and assess neurovascular structures. However, the interpretation of these complex images often requires highly trained specialists, and is subject to human error, high variability, and extended turnaround times.

AI based approaches have been proposed for automatic interpretation of medical images to accelerate decision making and support intervention, for example in cases where a a patient is suspected to be a victim of a stroke, thereby reducing time to treatment. However, such conventional AI based approaches for obtaining a continuous vessel segmentation have reduced robustness, especially in the presence of signal dropout, noise, vessel tortuosity, calcification, and proximity to bone or bifurcations. Further, such conventional AI based approaches for detecting large vessel occlusions (LVO) are not able to identify the exact location of the occlusion within vessel distribution models.

To address these challenges, a variety of methods have been proposed. In EP 4 160 529 A1, a method for tracing a vessel tree and for detecting LVO in medical imaging is disclosed. The method proposed therein employs a probabilistic approach to generate a tree of vessels from anatomical landmarks and vessel centerlines identified and/or determined in a medical image of vessels of a patient.

Deep learning has catalyzed substantial advancements in the fields of vessel tree segmentation, which is a critical step in constructing vascular trees, and the detection of neurovascular abnormalities. For the task of vessel tree segmentation, cutting-edge methodologies utilize architectures akin to U-Net to generate binary vessel masks from medical images, achieving impressively high Dice overlap. However, these techniques are often hindered by their inability to effectively generalize to various pathological scenarios, thereby restricting their applicability.

Despite significant advancements, a robust, efficient, and accurate pipeline capable of both accurately identifying vessels and accurately identifying abnormalities in a single system is still lacking.

### SUMMARY

It is an object of embodiments of the present invention to provide methods and devices for training and employing AI models for identifying vessel abnormalities with increased accuracies.

To solve at least this object, a training data preparation method, a medical data analysis method, devices for carrying out the methods as well as a computer program product are proposed according to the independent claims. Advantageous embodiments are the subject of the dependent claims.

In a training data preparation method, an input medical image of vessels of a patient is received. A vessel segmentation is determined from the input medical image. Anatomical landmarks in the vessel segmentation are identified and annotated to produce an annotated vessel segmentation. The annotated vessel segmentation is stored as training data.

Such annotated training data allows for improved training of AI models not only to recognize the vascular tree from a medical image but also to, at the same time, identify landmarks in the vascular tree.

In some embodiments, before storing the annotated vessel segmentation as training data, the method comprises inserting an abnormality into the vessel segmentation.

Training data that is augmented in this way yields AI models that are more robust when examining medical image data that comprises abnormalities.

In some embodiments, inserting an abnormality into the vessel segmentation comprises removing a part of the vessel segmentation to simulate an occlusion of a vessel.

Such training data yields AI models that are able to recognize and/or locate occlusions in medical images.

In some embodiments, inserting an abnormality into the vessel segmentation comprises adding a section to the vessel segmentation to simulate an aneurysm.

Such training data later yields AI models that are able to recognize and/or locate aneurysms in medical images.

In some embodiments, the method comprises training, using the stored training data, a first artificial intelligence model for generating vessel segmentations from input medical images and training, using the stored training data, a second artificial intelligence model for determining anatomical landmarks in input medical images, wherein the training steps are carried out assigning a higher weight to regions where an abnormality was inserted.

Thus, the trained artificial intelligence models will more precisely and reliably recognize abnormalities in medical image data.

An embodiment of a medical image data analysis method comprises receiving an input medical image of vessels of a patient; determining, by a first artificial intelligence model trained as described above, a vessel segmentation from the input medical image; determining, by a second artificial intelligence model trained as described above, anatomical landmarks of the vessels from the input medical image.

The thusly trained first artificial intelligence model and second artificial intelligence model provide a vessel segmentation and landmark detection of highly improved precision.

In some embodiments, the method comprises determining a semantic tree of vessels and determining a location where a part of the semantic tree of vessels is missing from the vessel segmentation and determining that location to be the location of an abnormality.

Such an automatic detection accelerates the process from taking a medical image to determining a diagnosis. If a large vessel occlusion is detected by this method, treatment can rapidly begin, potentially saving lives.

In some embodiments, the method comprises generating a surface model from the vessel segmentation; calculating a local vessel radius as a distance between a section of the surface model and the centerline for a multitude of sections; determining a location where a difference in local vessel radius between two sections exceeds a threshold as a location of an abnormality.

In this way, an aneurysm may be detected quickly and thus treatment begun immediately.

In some embodiments, a U-Net Segmentation Network is used as the first and/or the second artificial intelligence model, wherein the training data is prepared such that vascular landmark regions are labeled as foreground.

Such networks are particularly robust for detecting biological features and can be well trained to precisely detect landmark regions. Applying another U-Net to the landmark regions may produce very precise vascular segmentation in those areas.

In some embodiments, a U-Net network is used as the first and/or second artificial intelligence model, wherein the U-Net network is trained from the training data to detect objects of interest and, at the same time, perform at least one auxiliary task.

In this way, the same network may, for example, detect anomalies while also providing vessel segmentation.

In some embodiments, the method comprises determining a semantic tree of vessels and tracing a path along the semantic tree of vessels from an entry point to the abnormality.

It is understood that the method steps as disclosed above, below with respect to embodiments of this disclosure and/or as identified in the claims can be implemented in terms of dedicated processing means or by a processor suitable for carrying out the steps in a computer implemented fashion. Thus, the object is also solved by an apparatus comprising a device, at least one processor, mechanism, or means for carrying out the above-mentioned methods.

Furthermore, a computer program comprising instructions which, when the program is executed by a computer, cause the computer to carry out the steps of any the methods described above, is also proposed to solve the problems.

This will allow a computer to achieve the above-mentioned advantages of the method executed.

The computer program may be provided as or on computer-readable storage media comprising instructions which, when executed by a computer, cause the computer to carry out the steps of any of the above-mentioned methods.

In this way, the computer program may be provided advantageously.

### BRIEF DESCRIPTION OF THE DRAWINGS

Further embodiments and advantages may be gathered from the enclosed figures which schematically show embodiments of the present invention. In particular:

FIG. 1 shows a 3D representation of a semantic vascular tree;

FIG. 2 shows a schematic flow diagram of an embodiment of a method for preparing training data;

FIG. 3 shows 3D representations of annotated vessel segmentations;

FIG. 4 shows a schematic flow diagram of an embodiment of a method for preparing training data;

FIG. 5 shows a schematic flow diagram of an embodiment of a method for training an AI module;

FIG. 6 shows a schematic flow diagram of an embodiment of a method for determining an intervention path;

FIG. 7 shows example path planning results from CTA images; and

FIG. 8 shows an embodiment of an apparatus for training data preparation and/or for medical image data analysis.

### DETAILED DESCRIPTION

The present invention generally relates to methods and systems for vascular abnormality detection in medical imaging. Specifically, the present invention relates to methods and systems for AI assisted vessel segmentation and centerline detection. Also, the present invention relates to methods for preparing training data for at least one artificial neural network and to methods for training said artificial neural network.

Embodiments of the present invention are described herein to give a visual understanding of such methods and systems. A digital image is often composed of digital representations of one or more objects (or shapes). The digital representation of an object is often described herein in terms of identifying and manipulating the objects. Such manipulations are virtual manipulations accomplished in the memory or other circuitry/hardware of a computer system. Accordingly, it is to be understood that embodiments of the present invention may be performed within a computer system using data stored within the computer system.

Embodiments described herein provide for vascular abnormality detection in medical imaging. Embodiments described herein use semantic knowledge of anatomical landmarks of a vascular tree, combined with vessel centerlines identified using a deep learning model trained as described herein, to detect vascular abnormalities such as aneurysms and/or large vessel occlusions (LVO). Furthermore, embodiments described herein use such semantic knowledge to automatically locate LVO and aneurysms and compute 3D paths for intervention planning.

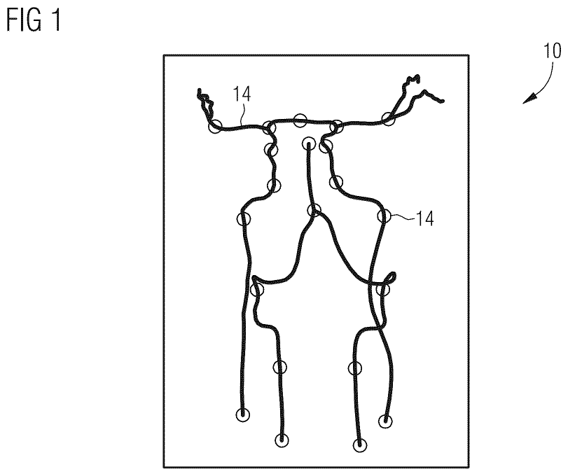

Virtually all humans have a vascular tree 10 that follows a common structure such as the one shown in FIG. 1. When a medical image is taken of the vascular system by a medical image capture device, such as a Computed Tomography Angiography (CTA) and Magnetic Resonance Imaging such as Magnetic Resonance Angiography (MRA), Time-of-Flight Magnetic Resonance Angiography (MRTOF) and post contrast T1-weighted MRI device, a 3-dimensional representation of the effect measured by the device is returned as a medical image. As such, the expression “image” is not restricted to two-dimensional representations. An image may be generally comprised of pixels (picture elements), wherein each pixel represents a physical value measured at a location associated with the pixel. Such a physical value may represent in particular, but not limited to, a brightness, color, reflectivity, degree of transmission or magnetic resonance intensity. In some image capture devices, such as in CT devices, no images in the traditional sense may be immediately produced by the diagnostic procedure. Rather, multiple items of raw data may be combined algorithmically to form or reconstruct the medical image. In such embodiments, a pixel in the resulting calculated image may not be associated with just one immediate surface or volume element of the recorded item or person but its value may be the result of a combination of multiple measuring points.

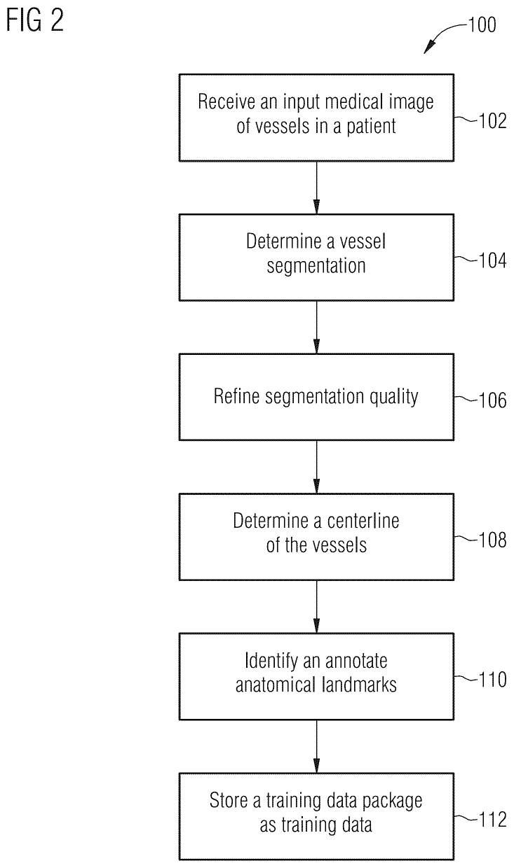

FIG. 2 shows a method 100 for generating training data for training a machine learning network from an input medical image, in accordance with one or more embodiments. The steps of method 100 may be performed by one or more suitable computing devices, such as, e.g., a general purpose computer.

At step 102 of the method, an input medical image of vessels in a patient, for example a medical image as described above, is received.

At step 104, an initial vessel segmentation is determined from the input medical image. In some embodiments, step 104 is carried out by application of a threshold-based method, in which pixels of the medical image that fall into a certain range of values are considered to be part of the vascular tree. Further such methods are known, such as the method described in initially mentioned EP 4 160 529 A1 which has the advantage of improved precision relative to the threshold-based method. Some U-Nets have been trained to determine vessel segmentation in specific pathological scenarios.

Also, some computer applications such as 3D Slicer have an interactive interface for configuring an automatic implementation of step 104, which generates a rough initial vessel segmentation. However, this initial segmentation often includes numerous false positives, which are non-vessel segmentations.

In step 106, the segmentation quality is refined.

In step 108, anatomical landmarks of the vessels are determined from the vessel segmentations. To this end, an AI model may be employed. As well, an extension to the 3D Slicer application may be employed, named “Vascular Modeling Toolkit” as published on (https://github.com/vmtk/SlicerExtension-VMTK), for automatic detection of anatomical landmarks. After automatic determination of the centerline, an operator may make modifications or corrections to the anatomical landmarks to improve their accuracy.

Other computer applications that allow similar automated anatomical landmark detection may exist and may be used for the purpose of refining step 108.

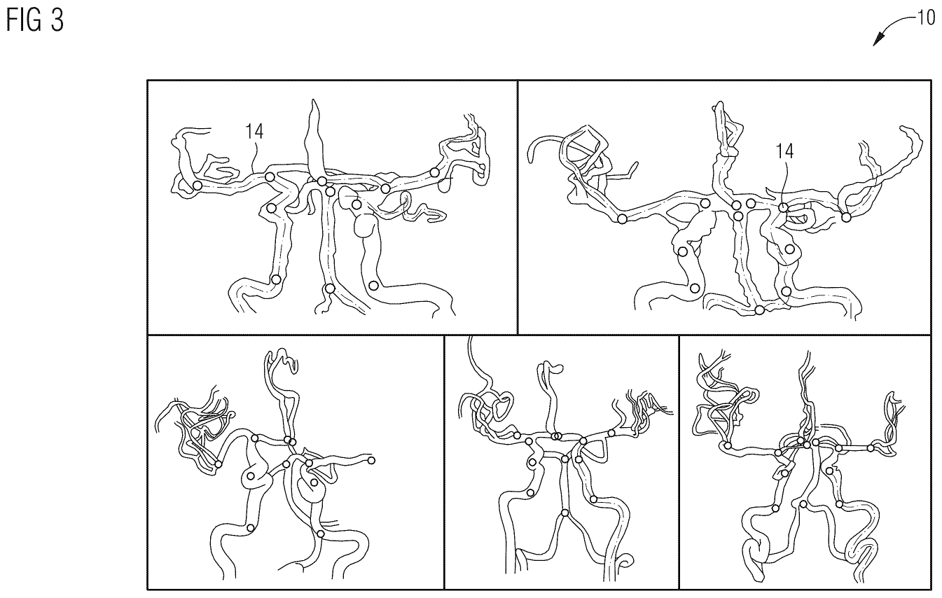

In step 110, vascular landmarks 14, such as shown in FIG. 3, may be identified and annotated in the vascular tree 10. Vascular or anatomical landmarks may comprise, among others, centerlines of vessels, the carotid frontal, carotid artery merge, middle cerebral artery, intracranial basilar artery branch and vertebral artery merge. Step 110 may also comprise use of the method described in EP4160529A1.

In step 112, a training data package is stored as training data. The training data package may comprise one or more of the following: The input medical image received in step 102, the vessel segmentation, the anatomical landmarks and/or the annotations, such as centerlines.

Appling method 100 to multiple input medical images yields a corpus of annotated training data.

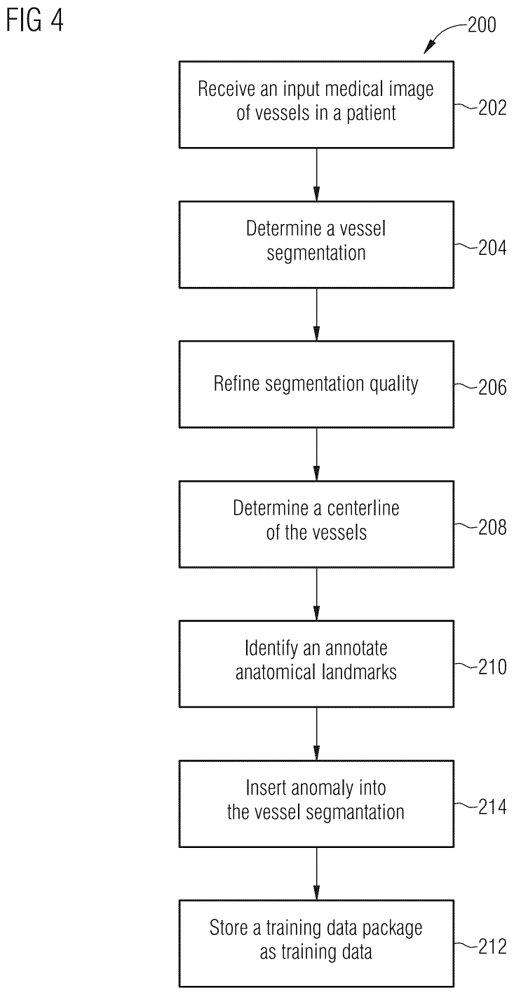

To improve detection accuracy, a method 200 as shown in FIG. 4 may be employed. Steps 102, 104, 106, 108, 110 and 112 of method 100 are equivalent to steps 202, 204, 206, 208, 210 and 212, respectively, of method 200.

However, method 200 comprises an additional step 214, in which an abnormality is inserted into the vessel segmentation. The abnormality to be inserted may be any possible abnormality known to occur.

For example, in some embodiments of step 214, a large vessel occlusion (LVO) may be inserted as an abnormality, in particular randomly inserted, into one of the segments on the tree. To this end, basically, a part of the vessel segmentation is removed to simulate an occlusion of a vessel since the vessel segmentation represents a blood-filled vessel.

As would be the case in a real-world LVO, the downstream segmentation and centerline annotation in the corresponding segment will be removed. No blood would flow through a real LVO, so this would simulate the cessation of blood flow. If the LVO segment chosen as the location of the random abnormality is the sole blood supply to the downstream segments of the vascular tree, the corresponding downstream segments will also be removed. To this end, a representation of a healthy vascular tree with downstream relations included may be provided.

For instance, if an LVO is inserted in the ICA, the downstream ICA segment will be removed but M1 and M2 will be retained as there are other sources of blood supply. However, if an LVO is inserted in M1, the rest of M1 along with M2 will be removed to simulate a realistic situation. In CTA, MRA or MRTOF scans, the intensity of the corresponding removed regions will be non-linearly transformed to match the intensity of the surrounding brain tissue.

In some embodiments of step 214, an aneurysm may be inserted, in particular randomly inserted, into the vessel segmentation. Since an aneurysm would make a vessel appear larger, a section is added to the vessel segmentation to simulate an aneurysm.

Aneurysms commonly appear as small surface lumps or restricted enlargements of the vascular tree. A potential method for detecting such abnormalities is to measure a local radius or distance to the centerline. However, due to the small size of aneurysms in the early phase of the disease, such methods may not be sufficiently sensitive due to mis-segmentation or shift of the automatic centerline.

Since the vessel segmentation represents the form of the physical vessel in a digital form, this form may be changed algorithmically. The aneurysm may, for example, be inserted into the vessel segmentation as a small lump on the surface of the vessel or a local enlargement. Both the size and location will be random. Although the vessel segmentation is modified, a possible centerline annotation is unchanged to allow the AI system to identify the true centerline in such pathological cases.

This will, in systems trained with the resulting training data, improve the detection of aneurysms, especially in early phases.

To prepare training data based on a medical input image from a CTA/MRA/MRTOF scan, the intensity of the inserted area will be set to the intensity of the attached vessel with a small amount of smoothing on the edge of the simulated lesion.

In some embodiments, a visual inspection step may be provided before step 212 of storing the annotated vessel segmentation data. In said visual inspection step, an operator may, for example, visually inspect the augmented vessel segmentation data to ensure their quality such that the insertion does not result in obviously incorrect results.

If an abnormality was inserted in step 214 or the input medical image received in step 102, 202 already comprised an abnormality, the location within the vascular segmentation and the type of the abnormality may be stored together with the training data package in step 112, 212.

At step 302 of a training method 300 as shown in FIG. 5, a first AI model is trained using the training data obtained by method 100 and/or method 200 to determine vessel segmentations from medical image data. At step 304, a second AI model is trained using the training data obtained by method 100 and/or method 200 to determine anatomical landmarks such as centerlines from medical image data.

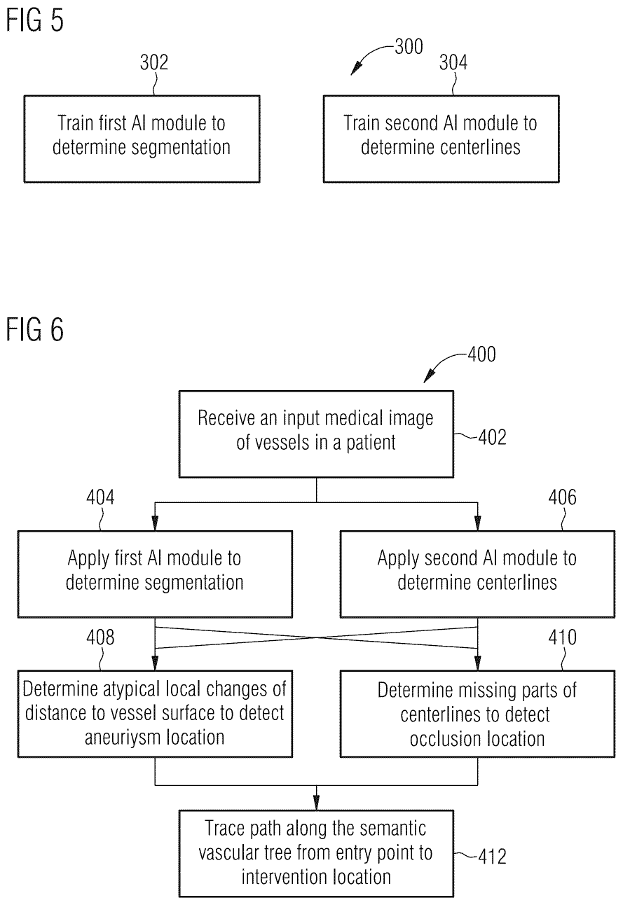

A medical analysis method 400 as shown in FIG. 6 comprises a step 402, in which an input medical image of vessels of a patient is received. In step 404, the input medical image is analyzed by the first AI model to determine vessel segmentations. In step 406, the input medical image is analyzed by the second AI model to determine anatomical landmarks such as centerlines.

In step 408, atypical changes of distance between a vessel surface and the associated centerline are determined. Where such atypical changes occur, an aneurysm is determined to be located. In some embodiments, a distance between a section of the vessel surface model and the centerline may be calculated for a multitude of sections. An abnormality may be determined to be located where a difference in distance between two sections exceeds a threshold.

In step 410, a semantic tree is constructed from the segmentations and/or the centerlines. Where parts of the semantic tree and/or the centerlines are missing, a large vessel occlusion is determined to be located.

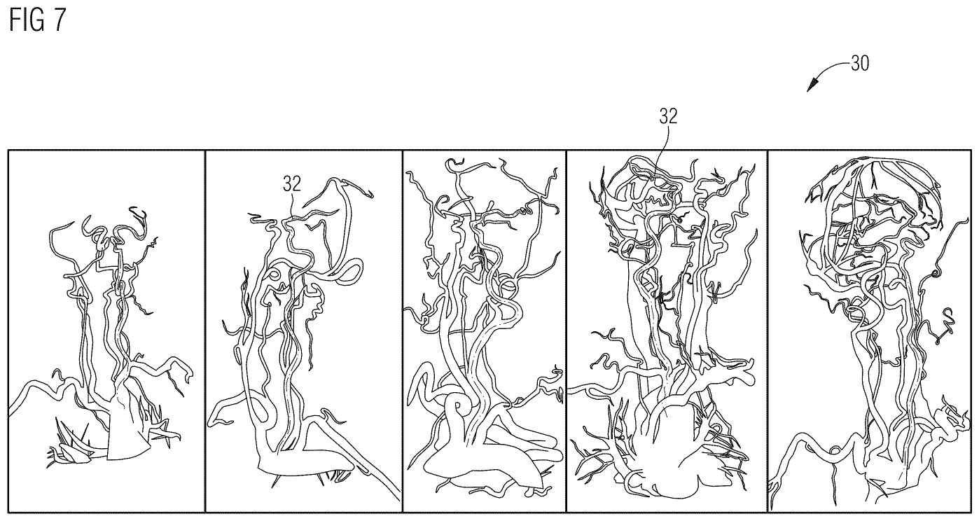

In step 412, a path is traced along the semantic vascular tree from a chosen entry point to the abnormality that was located in step 408 or step 410. Such as shown in FIG. 7, a visualization 30 of the vascular tree may be augmented with the intervention path 32, for example to prepare for an intervention to remove the abnormality. The intervention path 32 may later guide a chirurgic instrument through the vascular system of the patient from an entry point to the abnormality, thus simplifying the intervention and avoiding time-consuming mistakes.

In some embodiments, steps 408 and 410 may be replaced and/or integrated into steps 404 and 406 to leverage the augmented training data for detecting abnormalities.

In some such embodiments, a U-Net Segmentation Network may be applied as the first and/or the second AI model. Landmarks, such as those annotated in the training data, may be detected by said U-Net. The U-Net is trained such that the landmark detection problem is reformatted as a segmentation problem. In the training data, ground truth label maps with regions around the ground truth landmarks labeled as foreground are generated. The U-Net is then trained to produce segmentation of these landmark regions. The centerline and vessel segmentations can be input as additional features (channels) of the training data to the neural network to assist in the learning process.

In some further embodiments, a Retina U-Net as described in Bagcilar, Omer, et al. “Automated LVO detection and collateral scoring on CTA using a 3D self-configuring object detection network: a multi-center study.” Scientific Reports 13.1 (2023): 8834 is utilized as the first and/or second AI model. In this model, a U-Net-like network is trained to detect objects of interest while also performing auxiliary tasks (e.g., vessel segmentation). By jointly training the model with both tasks, it has been shown to effectively leverage the rich voxel-wise information from the vessel segmentation to improve detection accuracy.

The AI system proposed herein boasts several advantages over previous state-of-the-art methodologies, such as Robustness, Efficiency and/or Comprehensiveness.

Regarding robustness, the AI models for vessel segmentation and centerline annotation, trained using the proposed method, are expected to exhibit enhanced robustness in pathological situations. Said training method leads to higher sensitivity to pathological alterations, thereby facilitating more accurate detection of disease-related changes and augmenting diagnostic precision.

Regarding efficiency, given that the proposed approach is underpinned by deep neural networks, which is known for its efficiency during test time, the runtime of the proposed pipeline is anticipated to be small during deployment. This is of importance, as certain acute vascular conditions necessitate swift responses. In addition, time to prepare manual datasets for pipeline development will also be reduced using the proposed manual segmentation pipeline.

Regarding the last of the above-mentioned advantages, comprehensiveness, the output of the AI pipeline encompasses various stages of the clinical workflow, including the detection of abnormalities, vasculature visualization, and treatment planning, among others. This lends the system a versatile applicability, making it potentially suitable for a wide range of applications.

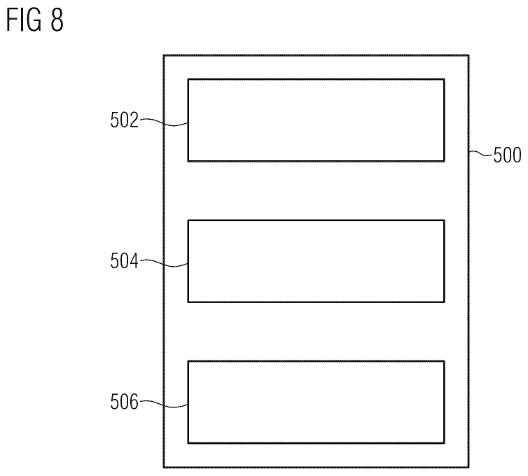

FIG. 8 displays an apparatus 500 that shows an embodiment of an apparatus for training data preparation and/or for medical image data analysis according to an embodiment of the present invention. The apparatus 500 is configured for executing a method for training data preparation and/or for medical image data analysis according to embodiments of the present invention.

The apparatus 500 can be or comprise a (personal) computer, a workstation, a virtual machine running on host hardware, a microcontroller, or an integrated circuit. As an alternative, the apparatus 500 can be a real or a virtual group of computers (the technical term for a real group of computers is “cluster”, the technical term for a virtual group of computers is “cloud”).

The apparatus 500 can comprise an interface 502, a computation unit 504 and a memory unit 506. An interface 502 can be a hardware interface or as a software interface (e.g., PCIBus, USB or Firewire). A computation unit 504 can comprise hardware elements and software elements, for example a microprocessor, a CPU (acronym for “central processing unit”), a GPU (acronym for “graphical processing unit”), a field programmable gate array (an acronym is “FPGA”) or an ASIC (acronym for “application-specific integrated circuit”). A computation unit 504 can be configured for multithreading, i.e., the computation unit can host different computation processes at the same time, executing the either in parallel or switching between active and passive computation processes. In particular, the computation unit 504 can be denoted as processor. The memory unit 506 can comprise one or more databases. Each of the interface 502, the computation unit 504 and the memory unit 506 can comprise several subunits which are configured to execute different tasks and/or which are spatially separated.

The apparatus 500 can be connected to one or more databases via a network. The network can be realized as a LAN (acronym for “local area network”), in particular a WiFi network, or any other local connection. Alternatively, the network can be the internet. In particular, the network could be realized as a VPN (acronym for “virtual private network”). Alternatively, the database can also be integrated into the apparatus 500, e.g., the database could be stored within the memory unit 506 of the apparatus 500. In this case the database is connected by an internal connection.

Systems, apparatuses, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computer and interact via a network. The client-server relationship may be defined and controlled by computer programs running on the respective client and server computers.

Systems, apparatus, and methods described herein may be implemented within a network-based cloud computing system. In such a network-based cloud computing system, a server or another processor that is connected to a network communicates with one or more client computers via a network. A client computer may communicate with the server via a network browser application residing and operating on the client computer, for example. A client computer may store data on the server and access the data via the network. A client computer may transmit requests for data, or requests for online services, to the server via the network. The server may perform requested services and provide data to the client computer(s). The server may also transmit data adapted to cause a client computer to perform a specified function, e.g., to perform a calculation, to display specified data on a screen, etc. For example, the server may transmit a request adapted to cause a client computer to perform one or more of the steps or functions of the methods and workflows described herein, including one or more of the steps or functions of FIG. 2, 4, 5 or 6. Certain steps or functions of the methods and workflows described herein, including one or more of the steps or functions of FIG. 2, 4, 5 or 6, may be performed by a server or by another processor in a network-based cloud-computing system. Certain steps or functions of the methods and workflows described herein, including one or more of the steps of FIG. 2, 4, 5 or 6, may be performed by a client computer in a network-based cloud computing system. The steps or functions of the methods and workflows described herein, including one or more of the steps of FIG. 2, 4, 5 or 6, may be performed by a server and/or by a client computer in a network-based cloud computing system, in any combination.

Systems, apparatus, and methods described herein may be implemented using a computer program product tangibly embodied in an information carrier, e.g., in a non-transitory machine-readable storage device, for execution by a programmable processor; and the method and workflow steps described herein, including one or more of the steps or functions of FIG. 2, 4, 5 or 6, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Designations such as “first”, “second” or “third” are used in this document only to distinguish similar but different items. They do not designate any kind of hierarchy.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

The foregoing Detailed Description is to be understood as being in every respect illustrative and exemplary, but not restrictive, and the scope of the present invention disclosed herein is not to be determined from the Detailed Description, but rather from the claims as interpreted according to the full breadth permitted by the patent laws. It is to be understood that the embodiments shown and described herein are only illustrative of the principles of the present invention and that various modifications may be implemented by those skilled in the art without departing from the scope of the present invention. Those skilled in the art could implement various other feature combinations without departing from the scope of the present invention.

Independent of the grammatical term usage, individuals with male, female or other gender identities are included within the term.

What is claimed is:

1. A computer-implemented training data preparation method, comprising:
receiving an input medical image of vessels of a patient;
determining a vessel segmentation from the input medical image;
identifying and annotating anatomical landmarks in the vessel segmentation to produce an annotated vessel segmentation; and
storing the annotated vessel segmentation as training data.

2. The method of claim 1, wherein before storing the annotated vessel segmentation as training data, the method comprises:
inserting an abnormality into the vessel segmentation.

3. The method of claim 2, wherein the inserting an abnormality into the vessel segmentation comprises:
removing a part of the vessel segmentation to simulate an occlusion of a vessel.

4. The method of claim 2, wherein the inserting an abnormality into the vessel segmentation comprises:
adding a section to the vessel segmentation to simulate an aneurysm.

5. The method according to claim 1, further comprising:
training, using the training data, a first artificial intelligence model for generating vessel segmentations from input medical images, and
training, using the training data, a second artificial intelligence model for determining anatomical landmarks in input medical images, wherein the training of the first artificial intelligence model and the training of the second artificial intelligence model are carried out assigning a higher weight to regions where an abnormality was inserted into the training data.

6. A medical image data analysis method, comprising:
receiving an input medical image of vessels of a patient;
determining, by a first artificial intelligence model, a vessel segmentation from the input medical image; and
determining, by a second artificial intelligence model, anatomical landmarks of the vessels from the input medical image, wherein
the first artificial intelligence model and the second artificial intelligence model are trained according to claim 5.

7. The method of claim 6, further comprising:
determining a semantic tree of vessels;
determining a location at which a part of the semantic tree of vessels is missing from the vessel segmentation; and
determining that the location is a location of an abnormality.

8. The method of claim 6, further comprising:
generating a surface model from the vessel segmentation;
calculating a local vessel radius as a distance between a section of the surface model and a centerline for a multitude of sections; and
determining, as a location of an abnormality, a location at which a difference in local vessel radius between two sections exceeds a threshold.

9. The method of claim 6, further comprising:
using a U-Net Segmentation Network as at least one of the first artificial intelligence model or the second artificial intelligence model, wherein the training data is prepared such that vascular landmark regions are labeled as foreground.

10. The method of claim 6, further comprising:
using a U-Net network as at least one of the first artificial intelligence model or the second artificial intelligence model, wherein the U-Net network is trained from the training data to detect objects of interest and, at the same time, perform at least one auxiliary task.

11. The method according to claim 7, further comprising:
tracing a path along the semantic tree of vessels from a surgical entry point to the abnormality; and
saving said path as a guidance for a chirurgical procedure.

12. An apparatus comprising:
at least one processor configured to perform the method of claim 1.

13. A non-transitory computer-readable storage medium comprising instructions that, when executed by a computer, cause the computer to perform the method of claim 1.

14. The method of claim 3, wherein the inserting an abnormality into the vessel segmentation comprises:
adding a section to the vessel segmentation to simulate an aneurysm.

15. The method according to claim 14, further comprising:
training, using the training data, a first artificial intelligence model for generating vessel segmentations from input medical images, and
training, using the training data, a second artificial intelligence model for determining anatomical landmarks in input medical images, wherein the training of the first artificial intelligence model and the training of the second artificial intelligence model are carried out assigning a higher weight to regions where an abnormality was inserted into the training data.

16. The method according to claim 2, further comprising:
training, using the training data, a first artificial intelligence model for generating vessel segmentations from input medical images, and
training, using the training data, a second artificial intelligence model for determining anatomical landmarks in input medical images, wherein the training of the first artificial intelligence model and the training of the second artificial intelligence model are carried out assigning a higher weight to regions where an abnormality was inserted into the training data.

17. The method of claim 7, further comprising:
generating a surface model from the vessel segmentation;
calculating a local vessel radius as a distance between a section of the surface model and a centerline for a multitude of sections; and
determining, as a location of an abnormality, a location at which a difference in local vessel radius between two sections exceeds a threshold.

18. The method according to claim 8, further comprising:
tracing a path along the semantic tree of vessels from a surgical entry point to the abnormality; and
saving said path as a guidance for a chirurgical procedure.

19. An apparatus comprising:
a memory storing computer-executable instructions; and
at least one processor configured to execute the computer-executable instructions to cause the apparatus to
receive an input medical image of vessels of a patient,
determine a vessel segmentation from the input medical image,
identify and annotate anatomical landmarks in the vessel segmentation to produce an annotated vessel segmentation, and
store the annotated vessel segmentation as training data.
